(19) United States
(12) Patent Application Publication
(10) Pub. No.: US 2013/0018384 A1
(43) Pub. Date: Jan. 17, 2013

(54) Medical Retrieval and Capture Device and Related Methods of Use

(71) Applicants: Gary Kappel, Action, MA (US); Larry Stanton, Burlington, MA (US); Ruth Cheng, Natick, MA (US); Ken Keene, Winchester, MA (US); Gerald Heller, Bedford, MA (US); Man Nguyen, West Roxbury, MA (US); William Shaw, Canbridge, MA (US)

(72) Inventors: Gary Kappel, Action, MA (US); Larry Stanton, Burlington, MA (US); Ruth Cheng, Natick, MA (US); Ken Keene, Winchester, MA (US); Gerald Heller, Bedford, MA (US); Man Nguyen, West Roxbury, MA (US); William Shaw, Canbridge, MA (US)

(21) Appl. No.: 13/547,928

(22) Filed: Jul. 12, 2012

Related U.S. Application Data
(60) Provisional application No. 61/508,160, filed on Jul. 15, 2011.

(51) Int. Cl. A61B 17/3205

(52) U.S. Cl. 606/111

(57) ABSTRACT

A medical device and related methods of use are provided for capture and removal of tissue from body lumens. The device includes an end-effector unit configured to capture tissue, having a distal end and a proximal end. The distal end and the proximal end form a substantially closed structure, and the distal end is more flexible than the proximal end. The device also includes a control member at the proximal end for holding and controlling the severing device.

### CROSS-REFERENCE TO RELATED APPLICATIONS

This application claims the benefits of priority under 35 U.S.C. §119-120 to U.S. Provisional Patent Application No. 61/508,160, filed Jul. 15, 2011, the entirety of which is incorporated herein by reference.

### FIELD OF THE INVENTION

Embodiments of this disclosure relate generally to medical devices and procedures. In particular, embodiments of the present disclosure relate to medical devices and procedures for capturing and retrieving material from inside a patient's body.

### BACKGROUND OF THE INVENTION

An endoscopic procedure typically involves indirect observation of a surgical field through an endoscope or similar device inserted through an incision or a natural anatomical opening. The endoscope generally takes the form of a long, flexible tube, including a light conductor along with one or more channels for inserting medical devices. Endoscopes provide platforms for employing numerous tools, such as devices to sever and remove objects from inside the body. The item to be retrieved can be a natural object, such as a blood clot, kidney stone, or polyp; or a foreign object, such as a coil, stent, or guidewire. Retrieval devices can take numerous forms, the most common being baskets, forceps, and snare loops.

The removal and retrieval of polyps is a frequent application for endoscopy, particularly during colonoscopy and similar procedures. In many cases, a snare loop may be used to facilitate the removal and retrieval of polyps or other material from with a body. An issue encountered during such procedures is the tendency for the snare to slip off the polyp, requiring the physician to reacquire the polyp and repeat the severing portion of the procedure. This problem often results from a lack of rigidity in the snare loop or low friction between the tissue and the snare loop. Increasing the rigidity of the snare loop, however, has been found to interfere with the function of the loop itself, which requires a certain degree of flexibility.

Thus, it would be desirable to increase the rigidity of a snare loop while retaining its flexibility, thus allowing consistent capture and removal of polyps and other targeted tissue.

### SUMMARY OF THE INVENTION

Embodiments of the disclosure provide a medical device and procedure for capturing material from body lumens.

In accordance with an aspect of the present disclosure, a medical device includes an elongate member having a proximal end, a distal end, and a lumen extending therebetween; a control member extending within the lumen of the elongate member, the control member having a distal end and a proximal end; and an end-effector disposed at the distal end of the control member, the end-effector including an expandable structure having a proximal portion and a distal portion, the proximal and distal portions defining a substantially continuous loop, wherein a portion of the end-effector is configured to cut through tissue, wherein the distal portion has a first flexibility and the proximal portion has a second flexibility different than the first flexibility.

Various embodiments of the medical device may include one or more of the following aspects: a plurality of fingers coupled to the proximal portion of the expandable structure, each of the plurality of fingers including an elongate structure having a flattened cross-sectional shape; at least one of the plurality of fingers includes a sharp edge configured to cut tissue; the fingers are configured to pivot relative to one another; at least one of the plurality of fingers is configured to bias the expandable structure against tissue; the plurality of fingers are configured to expand simultaneously with the expandable structure; the first flexibility is greater than the second flexibility; an overtube having a third flexibility greater than the first flexibility, the overtube coupled to the proximal portion of the expandable structure; the overtube includes a lumen, and at least the proximal portion of the expandable structure is disposed within the lumen; the fingers are configured to cauterize tissue; at least one of the plurality of fingers is configured to expand independently of the expandable structure; an outer surface of the plurality of fingers includes geometric features configured to facilitate gripping of tissues.

According to another embodiment, a medical device includes a sheath including a proximal end, a distal end, and a lumen; a control member extending within the lumen; and an end-effector disposed at a distal end of the control member, the end-effector including a plurality of overlying loops connected to one another by at least one link, each of the plurality of overlying loops configured to transition between compressed configuration and an expanded configuration, wherein the plurality of overlying loops are substantially parallel to one another in the expanded configuration.

In various embodiments, the end-effector may further include a balloon disposed proximate the plurality of loops.

A further aspect of the present disclosure provides a method for manipulating tissue from within a body of a patient. The method includes the step of introducing a medical device into the body. The medical device includes an elongate member having a proximal end, a distal end, and a lumen extending there between. The device further includes a control member extending within the lumen of the elongate member, the control member having a distal end and a proximal end. The device further includes an end-effector disposed at the distal end of the control member, the end-effector including an expandable structure having a proximal portion and a distal portion, the proximal and distal portions defining a substantially continuous loop, wherein at least a portion of the end-effector is configured to cut through tissue, wherein the distal portion has a first flexibility and the proximal portion has a second flexibility different than the first flexibility, and wherein the end-effector further includes a plurality of fingers coupled to the proximal portion of the expandable structure, each of the plurality of fingers including an elongate structure having a substantially flat and a greater cross-sectional dimension than a cross-sectional dimension of the distal portion of the expandable structure. The method further includes the steps of advancing the medical device to a desired location within the body, extending the end-effector out of the lumen, expanding the end-effector, and manipulating the tissue with the medical device.

In various embodiments, the method may further include one or more of the following aspects: the step of manipulating the tissue includes severing the tissue; severing tissue may include advancing the medical device to sever tissue with the proximal portion of the medical device; the step of manipulating tissue may include retrieving tissue with the distal portion of the medical device; at least one of the plurality of fingers configured to bias the expandable structure against tissue; an overtube having a third flexibility greater than the first flexibility, the overtube coupled to the proximal portion of the expandable structure.

Additional objects and advantages of the invention will be set forth in part in the description, which follows, and in part will be evident from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the disclosure and together with the description, serve to explain the principles of the disclosure. Wherever possible, the same reference numbers will be used throughout the drawings to refer to the same or like parts.

FIG. 1 illustrates a severing device in an open configuration according to an embodiment of the present disclosure.

FIG. 2 illustrates the severing device of FIG. 1 in a closed configuration.

FIG. 3 depicts a severing device according to another embodiment of the present disclosure.

FIG. 4 illustrates a severing device with two overlying parallel loops in an expanded configuration according to yet another embodiment of the present disclosure.

FIG. 5 depicts an alternate view of the severing device of FIG. 4 in a transient configuration.

FIG. 6 illustrates an alternate view of the severing device of FIG. 4 in a collapsed configuration.

FIG. 7 schematically illustrates an exemplary medical device with a severing device in an extended and expanded position at the distal end for retrieving and capturing tissue from body lumens.

FIG. 8 schematically illustrates an exemplary medical device with a severing device in a retracted and collapsed position.

FIG. 9 schematically illustrates the use of an exemplary severing device, according to an embodiment of the present disclosure.

### DESCRIPTION OF THE EMBODIMENTS

Reference will now be made in detail to embodiments of the disclosure, an example of which is illustrated in the accompanying drawings.

### Overview

The present disclosure provides a severing device for capturing and removing unwanted tissue growths. It should be noted that the severing device may actually perform the functions of both severing and capturing, but for convenience, the term “severing device” will be used throughout this application. The severing device may include an end-effector for capturing tissue and other target objects. The end-effector may include a distal end more flexible than its proximal end, and a control member connected to the device's proximal end for holding and controlling the severing device. The end-effector may form a snare loop, which can take an open configuration, generally describing a continuous or substantially continuous loop, which can be placed around or adjacent the target object, as well as a closed configuration in which a width dimension of the loop may be substantially reduced, thereby severing, or grasping a target object. The closed configuration may also be used to facilitate delivering the end-effector to a target location. As used in this disclosure, “distal” refers to a position or direction further from the user, and “proximal” refers to a position or direction opposite “distal” and closer to the user.

### Exemplary Embodiments

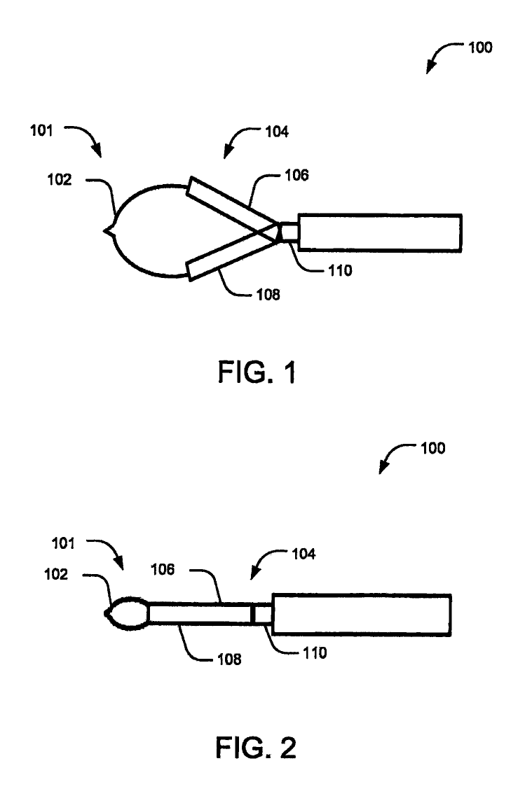

FIG. 1 depicts a severing device 100 in an open configuration according to an embodiment of the present disclosure. The severing device 100 includes an end-effector 101 extending distally from a control member 110. End-effector 101 is formed from a continuous loop of wire, including a flexible distal end 102 and a relatively stiff proximal end 104. Sizing and fabrication of the end-effector 101 lies well within the skill of those in the art and will not be discussed further here. In the open position depicted here, the severing device 100 can be placed over and around an object to be severed or grasped, such as a polyp, or a kidney stone, or a blood clot. The severing device 100 can be operated in conjunction with an endoscope or similar instrument (not shown).

In the illustrated embodiment, the stiffness of proximal end 104 is imparted by a pair of fingers 106, 108, pivotally mounted at the distal end of the control member 110 and extending along the proximal end 104 of end-effector 101. Fingers 106, 108 are elongated, generally rigid members, joined at a pivot point (not shown) at their proximal ends. As is known in the art, a spring or other suitable resilient means (not shown) biases the fingers 106, 108 in the open position shown in FIG. 1. The fingers 106, 108 impart stiffness to the proximal end 104 of end-effector 101 by being attached to the wire loop, either directly, as by welding or the like, or by design elements such as hooks or the like that fit over the wire. Design elements to be considered in selecting particular fingers 106, 108 include the length of the elements, dictated primarily by the desired size of the snare loop to be deployed by end-effector 101; the shape of the elongated portion, which can be generally planar, tubular, or rounded, as known in the art; the spring means, which can be a coil spring or other known resilient device; and the method of attaching the elongated portion to the wire loop, as discussed above.

Fingers 106, 108 can be formed of any suitable biocompatible material in art. The material can be selected from among those generally available to the art based on desired stiffness, resilience, and other properties of the elongated portion. Those factors, as well as compatibility with remaining portions of the medical device, my well within the skill of those in the art.

FIG. 2 illustrates the severing device 100 of FIG. 1 in a closed configuration. In this configuration, end-effector 101 has been almost completely retracted, allowing severing device 100 to grasp or sever a target object. Retracting end-effector 101 has the additional effect of urging the distal ends of fingers 106, 108 inward until the individual members of that element are folded together. The process by which the end-effector 101 is retracted is set out in some detail below, but it should be noted here that the retraction significantly reduces the size of the loop formed by the end-effector 101 to a small loop suitable for grasping or severing. Indeed, if desired, end-effector 101 can be completely retracted, and in one embodiment of severing device 100, both the fingers 106, 108 and end-effector 101 can be completely withdrawn into an endoscope or similar instrument (not shown). That configuration could prove useful for moving the unit through a patient's body lumens in preparation for use.

In alternate embodiments of severing device 100, fingers 106, 108 can be modified to improve functionality of the end-effector 101, or to accomplish additional operations. For example, inside edges of fingers 106, 108 may be provided with rough or serrated edges, or small projections, allowing the severing device 100 to grasp tissue firmly or retrieve severed tissue. Similarly, a cutting edge could improve severing capability, while cautery or radio frequency ablation capabilities could extend the capabilities of the severing device 100. It can readily be seen that operating the fingers 106, 108 in tandem smoothly changes the shape of end-effector 101 from the fully open configuration of FIG. 1 to the fully closed configuration of FIG. 2. The fingers 106, 108 may be linked with a pivot allowing the fingers 106, 108 to open and close so that fingers 106, 108 may be, e.g., operated as blades. Alternatively, finger 106 could be operated independently of finger 108, altering the shape of end-effector 101 from a generally symmetrical to a generally asymmetrical or elongated pattern, as might be needed in a particular situation to place the end-effector 101 in position to engage a particular target object.

Alternatively, rather than being oriented generally along the same axis as the control member 110, the fingers 106, 108 could be angled. That configuration would allow an operator to pre-load end-effector 101 against specific tissue, as might be seen in the course of removing a polyp. On the other hand, end-effector 101 and fingers 106, 108 could operate completely independently, rather than being attached. That configuration would allow the fingers 106, 108 to press adjacent tissue downward, providing counter-pressure to assist in engaging the target object. Further, one or more of fingers 106, 108 could be curved in one or more planes. For example, the fingers 106, 108 could be substantially bowed or ‘C’ shaped. In further embodiments, one or more of fingers 106, 108 may include angled features. Other variations will be apparent to those of skill in the art.

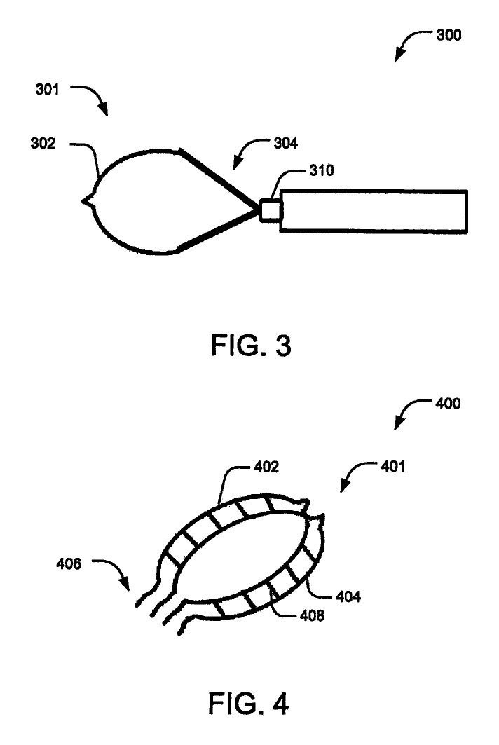

FIG. 3 depicts a severing device according to another embodiment of the present disclosure. The severing device 300 includes an end-effector 301 extending distally from a control member 310. As described in connection with FIG. 1, the end-effector 301 may be formed from a continuous or substantially continuous loop of wire. The embodiment shown here includes a flexible distal end 302 and a relatively stiff proximal end 304. Varying the stiffness along the length of the wire that forms an end-effector 301 can be accomplished in any suitable way known to the art. For example, fabricating proximal end 304 from wire thicker than that used to form distal end 302 would stiffen proximal end 304. Alternatively, a more malleable biocompatible material may be utilized for fabricating distal end 302. Further, a stiffening over-tube may be disposed over the proximal end 304 of the wire forming the end-effector 301. The over-tube may be fabricated with a less malleable biocompatible material than the material utilized for fabricating the end-effector 301. The over-tube may be formed in a number of cross-sections and dimensions, such as a rounded cross-section greater than the cross-section of the end-effector 301 wire.

Additionally, the proximal end 304 may be formed as a braid. In addition, sections of proximal end 304 and/or distal end 302 may be twisted to increase stiffness. Variably stiffened sections may be also created by annealing or cold working portions of proximal end 304 and distal end 302. Alternatively, a wire with a stiffer core may be utilized for fabricating proximal end 304. Similarly, a wire with a hollow core may be utilized to alter the stiffness of distal end 302. Further, proximal end 304 and/or distal end 302 may be fabricated from wires having different cross-sectional geometries or dimensions. For example, proximal section 304 may be fabricated with a wire having triangular or square cross-section and the distal end 302 may be fabricated from a wire with round cross-section. Further, sections of the end-effector 301 may be flattened to create flexibility in one plane and stiffness in another. Other suitable methods will be known to those in the art.

In all the embodiments discussed above in connection with FIGS. 1-3, control member 110 controls the end-effector 101 as well as provides a mounting location for that element. Control member 110 is carried at the distal tip of an endoscopic device (not shown), as known in the art, and it may include an actuation wire or cable (not shown), which in turn may be connected to an actuation handle (not shown), for manipulation by an operator. When deployed on an endoscopic device, control member 110 may assist the user in advancing the severing device 100 to the vicinity of tissue to be severed or retrieved. Those of skill in the art will be able to choose manufacturing methods for control member 110, along with suitable biocompatible material, such as nitinol, stainless steel, elgiloy tantalum, or polyimide.

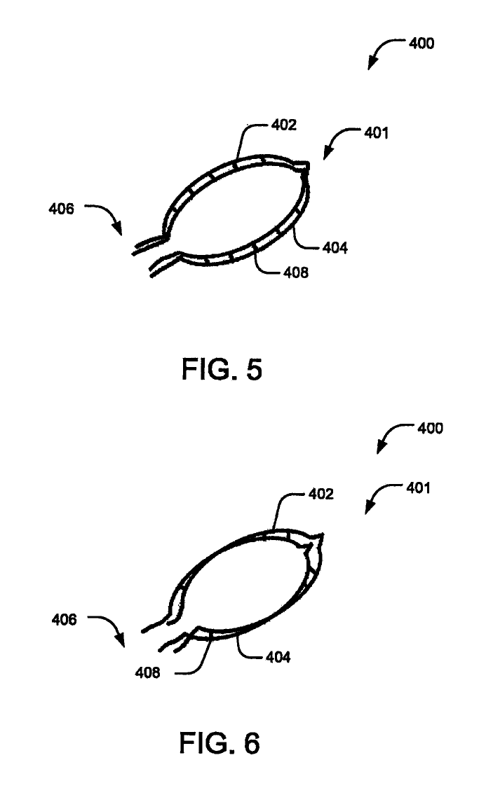

FIGS. 4-6 illustrate an alternative severing device having two overlying parallel loops that provide an end-effector having considerably greater thickness than those shown in FIGS. 1-3. Here, “thickness” is used to designate a direction perpendicular to the plane of end-effector 401. As shown here, severing device 400 includes an end-effector 401 formed from two overlying loops 402, 404. A series of links 408 extends between loops 402, 404. Each of the links 408 connects at one end to loop 402 and at the other end to loop 404, such that links 408 can pivot at least in a direction parallel to the longitudinal axis of the loops 402, 404. Those in the art can choose from among a number of joining methods to provide the desired pivotal motion, such as a universal joint, a flexing joint, or a flexible adhesive connection. Factors relevant to the choice of a particular joint include the degree of flexibility desired for particular applications, the likelihood of material fatigue affecting performance, and other considerations known to those in the art.

FIGS. 4-6 illustrate the manner in which the depicted embodiment provides an adjustable thickness of end-effector 401. As can be seen, the thickness of end-effector 401 depends upon the relative position of loops 402 and 404. An operator can adjust those positions by retracting and extending control wires (not shown) attached of proximal ends 406 of loops 402, 404. Maximum thickness, shown in FIG. 4 occurs when the relative loop positions are adjusted so that links 408 stand generally perpendicular to the plane of end-effector 401. In that position, loop 402 is vertically spaced above loop 404 at a distance determined by links 408. Minimum thickness, conversely, shown in FIG. 6, is achieved by adjusting the relative positions of loops 402 and 404 so that they generally lie in mutual contact, with links 408 generally lying in the plane of the two loops 402, 404.

The embodiment of FIGS. 4-6 provides an end-effector 401 having greater stiffness and greater thickness then end-effector 101 discussed above in connection with FIGS. 1-3. The collapsible framework formed by the loops 402, 404 and the set of links 408, provides increased stiffness and thickness. Stiffness is achieved because the individual wires forming loops 402, 404 are restrained from movement by the links 408. Further, this embodiment can be useful for grasping a target object such as polyps or embedded calculi, where the operator desires to minimize the pressure exerted against the target object by the wire loop of end-effector 401. That objective can be achieved by employing multiple wire loops in the end-effector 401, which in turn is achieved as shown in FIGS. 4-6. Here, the length of links 408, and thus the overall stiffness and thickness of end-effector 401, will be selected based on the specific target object to be grasped.

Loops 402 and 404 may be controlled together or, independently. In one implementation, one loop is fixed and the other is movable. In a further alternative design, links 408 can be formed in an ‘X’ pattern. In another design, the links may be formed in a ‘V’ pattern. Another design could employ an expansion member such as an inflatable balloon, positioned around top loop 402. In that configuration, end-effector 401 is deployed near the target site (not shown) and then the balloon inflates, which expands top loop 402 to an open configuration. Moreover, the balloon imparts stiffness to top loop 402, and the set of links 408 imparts stiffness to bottom loop 404. Alternatively, a balloon could be provided spanning the structure of loops 402, 404, lengthwise or in combination. Other self-expanding means could be employed with top loop 402 to achieve the requisite stiffness, such as spring means known in the art.

Control of the multiple-loop embodiments of FIGS. 4-6 can be effected with a split push-pull finger mechanism (not shown) or other suitable control element known in the art. Modifying a conventional split push-pull finger spool arrangement (with, e.g., finger holes in the handle, not shown), a bottom finger hole could operate bottom loop 404 and a top finger hole could operate top loop 402. Alternative capabilities discussed above, such as cautery or radio frequency ablation can likewise be applied to the embodiments of FIGS. 4-6 if desired. Constructional details of the end-effector 401 are generally identical to those set out above and will not be covered further here.

One implementation of the present disclosure provides an end-effector 401 coated with an anti-bacterial covering to inhibit bacterial growth on its surface. The antibiotic coating may contain an inorganic antibiotic agent, disposed in a polymeric matrix that adheres the antibiotic agent to the end-effector 401 surface. Further, a drug-releasing coating may also be applied to the outer surface of the end-effector 401, assisting in delivery of drugs to the severing site. In another alternative, imaging markers may be applied to the end-effector surface, to assist in locating the end-effector 401 within the body. Radiopaque, sonoreflective, or any other suitable markers may be employed.

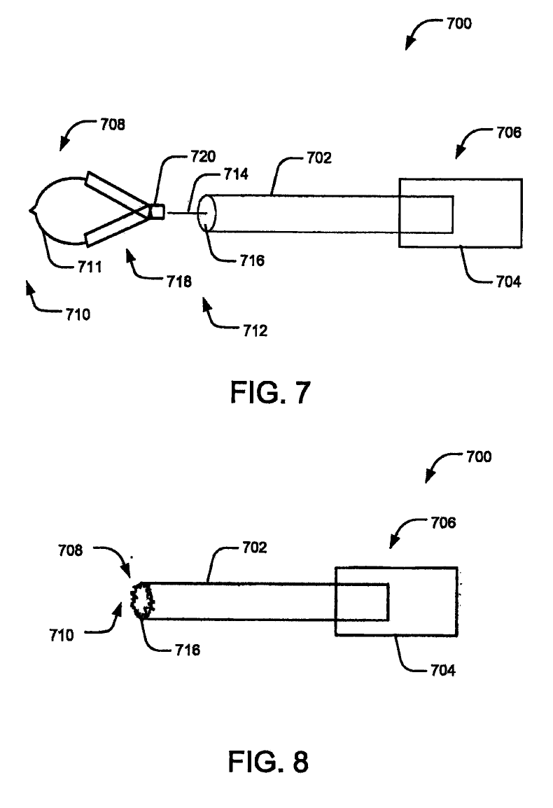

FIGS. 7-9 schematically illustrate a method of using the medical retrieval and severing device of the present disclosure. As seen in FIG. 7, an exemplary medical device 700 may include a conventional endoscope, sheath, guide tube, or introducer having a severing device 708 at its distal end for retrieving and capturing tissue or other target objects from body lumens. Severing device 708 also may be a discrete instrument configured to be introduced through a lumen of another instrument. A severing device 708 extends through lumen 716 formed in sheath 702. A connecting member 714 operatively connects the severing device 708 to a control device 704, which may be an actuation handle, an automated hand, or other conventional device for receiving and transmitting user input in an endoscope.

Severing device 708 includes an end-effector 710 extending from a control member 720, at the distal end of connecting member 714. In the illustrated embodiment, end-effector 710 is formed as a continuous loop of wire, including a flexible distal end 711 and a proximal end with stiffness imparted by a pair of fingers 718. In some embodiments, fingers 718 may be elastic or spring-like. As explained more fully below, stiffening the proximal end of the severing device 708 assists in holding end-effector 710 fully open during tissue capture, easing the operator's task of placing the end-effector 710 over a target object. Alternatively, severing device 708 may be stiffened in accordance with any one of the embodiments described in FIGS. 1-4.

End-effector 710 may assume the fully extended, or open, position shown in FIG. 7, or it may assume the retracted position shown in FIG. 8. There, end-effector 710 is retracted into lumen 716. In this configuration, an operator can easily advance sheath 702 through a patient's body lumen to a desired body location with minimum probability of damaging tissue along the way.

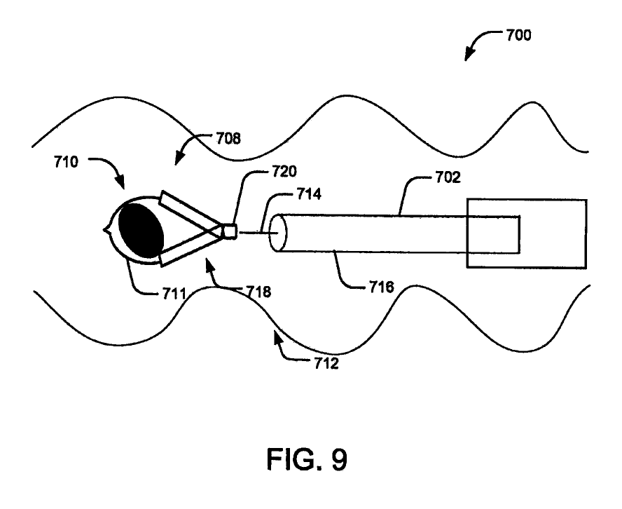

FIG. 9 illustrates the situation in which sheath 702 is positioned at the desired body location. End-effector 710 may be advanced out of sheath 702 into the fully open configuration for severing or capturing a target object. The target object could be a tissue growth, such as a polyp, that must be both severed and retrieved, or an object such as a kidney stone that must be retrieved. In the fully open position, fingers 718 are biased toward an open position, as explained above, ensuring that end-effector 710 presents the largest possible target acquisition area.

The operator employs control device 704 to effect the severing operation. Once the target object is encircled by end-effector 710, the operator may manipulate control member 720 to retract severing device 708 into the lumen 716, moving the end-effector 710 toward the orientation shown in FIG. 2, with the fingers 718 folded together and the distal end 711 of the end-effector 710 retracted. If the task requires severing tissue, the operator fully retracts the end-effector 710, so that the wire acts to sever the target tissue. In situations where the task is to retrieve an object, the operator retracts the end-effector 710 until the object is securely grasped. A severing operation, of course, may require that the severed tissue be retrieved, in which case the operator follows the severing of operation with a retrieval step, as described. Upon completing the task, the operator retracts sheath 702 from the patient's body. It should be apparent that the severing and retrieval device of the present disclosure is useful to perform surgical and diagnostic procedures in a wide variety of bodily locations. For example, removal of polyps detected during a routine colonoscopy could quickly be accomplished using the method discussed above. Additionally, stones or mineral deposits can be engaged and retrieved from a variety of bodily lumens, such as ureters, bladder, or the urethra.

In another embodiment, severing device 708 may be used to assist in the severing of tissue. For example, in embodiments where fingers 718 may operate as blades, severing device 708 may be advanced into a patient's body to partially or fully resect tissue. In particular, fingers 718 may be actuated (e.g., opened and closed) by a pull wire (not shown) or by advancing sheath 702 over the fingers 718 to close them. Furthermore, fingers 718 may be conductive so as to perform electrical resection procedures. In conductive embodiments, however, fingers 718 may not be limited blade or blade-like shapes. In some embodiments, the end-effector 710 may be monopolar. In other embodiments, however, end-effector 710 may be bipolar, i.e., each of fingers 718 may be an opposite pole. In FIGS. 4-6, for example, each loop may have opposite pole. On the other hand, the connecting links may be insulative. The end-effector 710 may further include an electrical adapter at a proximal portion, and may be configured to conduct electricity to fingers 718 through the actuator wire or through a separate conductor. These and other procedures can be accomplished within the scope of the present disclosure.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
an elongate member having a proximal end, a distal end, and a lumen extending therebetween;
a control member extending within the lumen of the elongate member, the control member having a distal end and a proximal end; and
an end-effector disposed at the distal end of the control member, the end-effector including an expandable structure having a proximal portion and a distal portion, the proximal and distal portions defining a substantially continuous loop, wherein at least a portion of the end-effector is configured to cut through tissue, wherein the distal portion has a first flexibility and the proximal portion has a second flexibility different than the first flexibility.

2. The device of claim 1, further comprising a plurality of fingers coupled to the proximal portion of the expandable structure, each of the plurality of fingers including an elongate structure having a flattened cross-sectional shape.

3. The device of claim 2, wherein at least one of the plurality of fingers includes a sharp edge configured to cut tissue.

4. The device of claim 2, wherein the fingers are configured to pivot relative to one another.

5. The device of claim 2, wherein at least one of the plurality of fingers is configured to bias the expandable structure against tissue.

6. The device of claim 2, wherein the plurality of fingers are configured to expand simultaneously with the expandable structure.

7. The device of claim 1, wherein the first flexibility is greater than the second flexibility.

8. The device of claim 1, further including an overtube having a third flexibility greater than the first flexibility, the overtube coupled to the proximal portion of the expandable structure.

9. The device of claim 8, wherein the overtube includes a lumen, and at least the proximal portion of the expandable structure is disposed within the lumen.

10. The device of claim 2, wherein the fingers are configured to cauterize tissue.

11. The device of claim 2, wherein at least one of the plurality of fingers is configured to expand independently of the expandable structure.

12. The device of claim 2, wherein an outer surface of the plurality of fingers includes geometric features configured to facilitate gripping of tissue.

13. A medical device, comprising:
a sheath including a proximal end, a distal end, and a lumen;
a control member extending within the lumen; and
an end-effector disposed at a distal end of the control member, the end-effector including a plurality of overlying loops connected to one another by at least one link, each of the plurality of overlying loops configured to transition between compressed configuration and an expanded configuration, wherein the plurality of overlying loops are substantially parallel to one another in the expanded configuration.

14. The device of claim 13, wherein the end-effector further includes a balloon disposed proximate the plurality of loops.

15. A method for manipulating tissue from within a body of a patient, the method comprising:
introducing a medical device into the body, the medical device comprising:
an elongate member having a proximal end, a distal end, and a lumen extending there between;
a control member extending within the lumen of the elongate member, the control member having a distal end and a proximal end;
an end-effector disposed at the distal end of the control member, the end-effector including an expandable structure having a proximal portion and a distal portion, the proximal and distal portions defining a substantially continuous loop, wherein at least a portion of the end-effector is configured to cut through tissue, wherein the distal portion has a first flexibility and the proximal portion has a second flexibility different than the first flexibility, and wherein the end-effector further includes a plurality of fingers coupled to the proximal portion of the expandable structure, each of the plurality of fingers including an elongate structure having a substantially flat cross-sectional shape;
advancing the medical device to a desired location within the body;
extending the end-effector out of the lumen;
expanding the end-effector; and
manipulating the tissue with the medical device.

16. The method of claim 15, wherein manipulating the tissue includes severing the tissue.

17. The method of claim 16, wherein severing the tissue includes first advancing the medical device to sever tissue with the proximal portion of the medical device.

18. The method of claim 16, wherein manipulating the tissue includes retrieving the tissue with the distal portion of the medical device.

19. The method of claim 15, wherein at least one of the plurality of fingers is configured to bias the expandable structure against tissue.

20. The method of claim 15, an overtube having a third flexibility greater than the first flexibility, the overtube coupled to the proximal portion of the expandable structure.
